(19) United States
(12) Patent Application Publication
(10) Pub. No.: US 2006/0009485 A1
(43) Pub. Date: Jan. 12, 2006

(54) Method of reprocessing quaternary ammonium-containing neuromuscular blocking agents

(75) Inventors: Oded Friedman, Yehiel (IL); Oded Arad, Rehovot (IL); Tamir Fizitzki, Beer Sheva (IL); Josef Manasku, Omer (IL); Joseph Kaspi, Givatayim (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: CHEMAGIS LTD, Bnei Brak (IL)

(21) Appl. No.: 11/159,891

(22) Filed: Jun. 23, 2005

(51) Int. Cl. A01N 43/42; A61K 31/47

(52) U.S. Cl. 514/308

(57) ABSTRACT

Provided is a method for reprocessing neuromuscular blocking agents containing a quaternary ammonium salt, e.g., Rocuronium bromide, using a novel dealkylation method. The process is effective in obtaining a highly pure product from a contaminated starting material by heating, optionally in the presence of an organic solvent, to produce a dealkyated product. The dealkylated product is purified, e.g., by crystallization, and converted by any known method to a stable, highly-pure neuromuscular blocking agent.

### FIELD OF THE INVENTION

The present invention relates to a process for reprocessing neuromuscular blocking agents containing one or more quaternary ammonium groups, such as Rocuronium bromide, using a novel dealkylation method.

### BACKGROUND OF THE INVENTION

Neuromuscular blocking agents that contain one or more quaternary ammonium functional groups (such as Tubocurarine chloride, Atracurium besylate, and certain steroidal neuromuscular blocking agents such as Pancuronium bromide, Vecuronium bromide, and Rocuronium bromide,) have muscle paralyzing activity similar to the alkaloid curare or d-tubocurarine. Such neuromuscular blocking agents interrupt the transmission of nerve impulses at the skeletal neuromuscular junction. They can be of two types, competitive, stabilizing blockers (neuromuscular nondepolarizing agents) or noncompetitive, depolarizing agents (neuromuscular depolarizing agents). Both prevent acetylcholine from triggering the muscle contraction and are used as anesthesia adjuvants in the operating theatre for aiding intubation i.e. relaxation of vocal cords, jaw muscles etc. and also for surgery i.e. providing generalized muscle relaxation, as relaxants during electroshock, in convulsive states, etc. Typically, therapy is performed by i.v. administration of a suitable dosage form.

1-[(2β, 3α, 5α, 16β, 17β)-17-acetoxy-3-hydroxy-2-(4-morpholinyl)-androstan-16-yl-]-1-(2-propenyl)pyrrolidinium bromide, also known by the name Rocuronium bromide, an exemplary steroidal neuromuscular blocking agent, has the structural formula I:

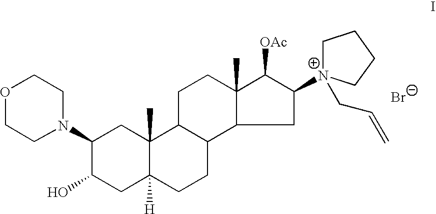

### Rocuronium Bromide

Presently, Rocuronium bromide is available commercially under the brand names Esmeron® and Zemuron®. Rocuronium bromide and the intermediates thereof are described specifically in U.S. Pat. No. 4,894,369 to Sleigh et al. and generally in a paper by Zoltan et al., Current Medicinal Chemistry, 9(16), 1507-1536, 2002. The synthesis of Rocuronium bromide is described in example 23 of U.S. Pat. No. 4,894,369, wherein it is obtained by reacting 2-propenyl bromide with (2β, 3α, 5α, 16β, 17β)-17-acetoxy-3-hydroxy-2-(4-morpholinyl)-16(1-pyrrolidinyl)-androstane having the structural formula II: 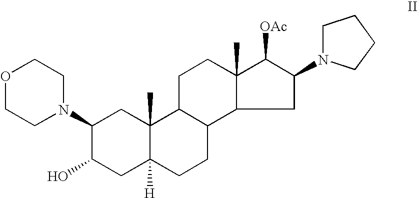
in dichloromethane, followed by column chromatography and precipitation of the pure product from a mixture of dichloromethane and diethyl ether.

While quaternary ammonium-containing neuromuscular blocking agents can be prepared synthetically, there are significant challenges associated with purifying them on a commercial scale. For instance, Rocuronium bromide is amorphous and very difficult to precipitate or crystallize. Furthermore, Rocuronium bromide has a tendency to retain organic solvents. Consequently, isolating Rocuronium bromide by precipitation would not necessarily overcome the problems associated with purification, since the product would retain the impurities left in the solution during precipitation. This is particularly problematic when off-grade batches are produced, which happens from time to time in the production of Rocuronium bromide. The off-grade batches are contaminated with organic impurities, inorganic impurities, and/or foreign matter at levels high enough to render such batches practically useless from a commercial standpoint, particularly in view of the fact that Rocuronium bromide is very difficult to crystallize. While it may be possible to chromatographically remove at least some production-related impurities, purification via column chromatography is very tedious and problematic for industrial use.

Hence there are presently no convenient, industrially viable methods for purifying Rocuronium bromide. Accordingly, there is a need for a practical method of recovering highly pure Rocuronium bromide, as well as other quaternary ammonium-containing neuromuscular blocking agents, from contaminated production batches on an industrial scale. The present invention provides such a method as will be apparent from the description of the invention provided herein.

### BRIEF SUMMARY OF THE INVENTION

The present invention provides a process for preparing a neuromuscular blocking agent, preferably Rocuronium bromide. An exemplary method of the present invention comprises dealkylating a Rocuronium bromide, to produce a dealkylated product. The dealkylation of Rocuronium bromide can be performed by heating in the absence of a solvent (neat) or in the presence of a suitable solvent. The dealkylated product can be conveniently purified to produce a purified dealkylated product, which is converted into highly pure Rocuronium bromide.

### DETAILED DESCRIPTION OF THE INVENTION

The present invention is predicated on the surprising and unexpected discovery that highly pure Rocuronium bromide can be obtained from contaminated production batches by dealkylating to produce a dealkylated product, purifying the dealkylated product to obtain a purified dealkylated product, and converting the purified dealkylated product into substantially pure Rocuronium bromide. The method of the present invention overcomes the problems and difficulties associated with the state of the art methods for purifying Rocuronium bromide, by providing a novel dealkylation process that is effective in obtaining a highly pure material by reprocessing. This simple and straightforward dealkylation enables reprocessing unstable amorphous Rocuronium bromide via its stable crystalline precursor II, to obtain a highly-pure product.

The reaction of dealkylation and specifically deallylation of quaternary ammonium groups is documented in the literature; however, the dealkylation of Rocuronium does not appear to have ever been reported. Dealkylation of quaternary ammonium halides by pyrolysis gave low yields along with decomposition products (V. Meyer et al, Ber., 8, 233, 1875). The use of reagents with high nucleophilicity along with lower reaction temperatures has resulted in less degradation (R. O. Hutchins et at, J. Org. Chem., 43, 2559, 1978). Several high-temperature dealkylations of quaternary ammonium halides have been reported. For example dealkylation may be achieved by heating the quaternary ammonium compound to 200° C. (H. Katayama et al, Chem. Pharm. Bull. 26,(7), 2027-2035, 1978), or by heating a solution of the compound in glycerin-water solvent mixture at 140° C. for 2-4 hours (H. Katayama et al, Chem. Pharm. Bull. 29,(9), 2465-2477, 1981), or by heating a solution of the compound in thiophenol to 70-90° C. for 3.5-10 hours (T. Kametani et al, J. Med. Chem., 12, 694-696, 1969).

The use of thiophenol or glycerin can be problematic when used on an industrial scale, however. Thiophenol is relatively toxic, can be explosive when mixed with air, and has a repulsive odor. Glycerin also is not particularly suitable for use on an industrial scale in view of its very high boiling point of 290° C. The method for dealkylating Rocuronium bromide in accordance with the present invention is particularly advantageous in that it does not require thiophenol or glycerin, or other specific nucleophilic reagents or solvents that may be unsuitable for use on an industrial scale. In a preferred embodiment, the present invention provides a process for obtaining substantially pure Rocuronium bromide from contaminated Rocuronium bromide production batches. In accordance with the present invention, the contaminated Rocuronium bromide can be dealkylated thermally (neat or in the presence of a solvent), e.g., by heating, to produce a dealkylated product, which can be readily purified on an industrial scale. The purified dealkylated product is reconverted into Rocuronium bromide, to produce highly pure Rocuronium bromide. The dealkylation reaction surprisingly and unexpectedly can be carried out in the absence of dealkylating agents, such as a nucleophilic reagents or additives that have been used in the art as dealkylating reagents. It is also surprising that Rocuronium bromide can be dealkylated, e.g., by heating in an organic solvent, without dealkylating reagents, to produce mostly dealkylation rather than decomposition.

In one embodiment, contaminated Rocuronium bromide is N-dealkylated by heating, either without solvent or in an organic solvent solution, to produce precursor II with minimal amounts of (2β, 3α, 5α, 16β, 17β)2-(4-morpholinyl)-16(1-pyrrolidinyl)-androstane-3,17-diol, having the structural formula III, and 1-[(2β, 3α, 5α, 16β, 17β)-2-(4-morpholinyl)-androstan-16-yl-]-1-(2-propenyl)-pyrrolidinium bromide-3,17-diol having the structural formula IV.

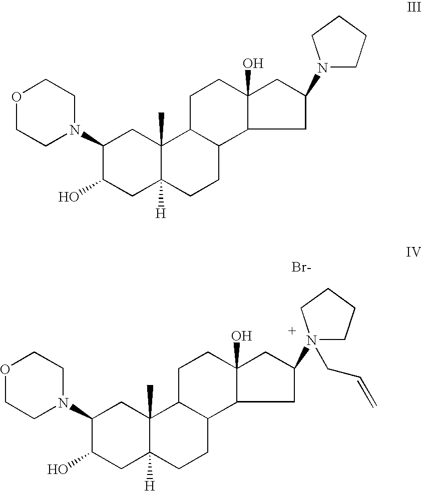

The obtained product (2β, 3α, 5α, 16β, 17β)-17-acetoxy-3-hydroxy-2-(4-morpholinyl)-16(1-pyrrolidinyl)-androstane II can be purified by crystallization and used to synthesize highly-pure Rocuronium bromide. The method of the present invention is particularly useful for recovering highly pure Rocuronium bromide from contaminated or off-grade production batches.

An exemplary process of the present invention is illustrated below in Scheme 1.

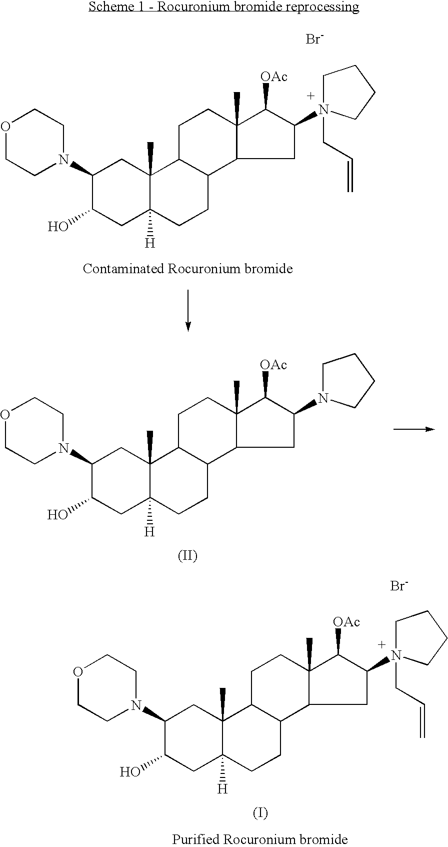

In a preferred embodiment, the present invention provides a method of reprocessing impure Rocuronium bromide comprising:
a) heating the impure Rocuronium bromide, optionally in the presence of an organic solvent, at a temperature effective to dealkylate the Rocuronium bromide, to produce (2β, 3α, 5α, 16β, 17β)-17-acetoxy-3-hydroxy-2-(4-morpholinyl)-16(1-pyrrolidinyl)-androstane II;
b) precipitating the (2β, 3α, 5α, 16β, 17β)-17-acetoxy-3-hydroxy2-(4-morpholinyl) 16(1-pyrrolidinyl)-androstane II, to obtain a purified product;
c) optionally crystallizing the (2β, 3α, 5α, 16β, 17β)-17-acetoxy-3-hydroxy-2-(4-morpholinyl) 16(1-pyrrolidinyl)-androstane II;
d) converting the purified (2β, 3α, 5α, 16β, 17β)-17-acetoxy-3-hydroxy-2-(4-morpholinyl)-16(1-pyrrolidinyl)-androstane II to Rocuronium bromide; and
e) recovering substantially pure Rocuronium bromide as a stable, non-hygroscopic solid.

In accordance with the present invention, the process for dealkylating Rocuronium bromide preferably comprises the steps of:
a) heating contaminated Rocuronium bromide, optionally in the presence of an organic solvent, at a temperature effective to dealkylate the Rocuronium bromide, to produce (2β, 3α, 5α, 16β, 17β)-17-acetoxy-3-hydroxy-2-(4-morpholinyl)-16(1-pyrrolidinyl)-androstane II;
b) adding water and an organic solvent, mixing the layers, and separating the organic layer, to obtain a solution of (2β, 3α, 5α, 16β, 17β)-17-acetoxy-3-hydroxy-2-(4-morpholinyl)-16(1-pyrrolidinyl)-androstane II in the organic layer;
c) drying the organic layer; and
d) evaporating the organic layer to dryness, optionally at an elevated temperature, to isolate the (2β, 3α, 5α, 16β, 17β)-17-acetoxy-3-hydroxy-2-(4-morpholinyl)-16(1-pyrrolidinyl)-androstane II.

In accordance with the present invention, the contaminated Rocuronium bromide may be dealkylated by heating in the absence or in the presence of a solvent (e.g., an organic solvent) to obtain (2β, 3α, 5α, 16β, 17β)-17-acetoxy-3-hydroxy-2-(4-morpholinyl)-16(1-pyrrolidinyl)-androstane II. As used herein, the term “solvent” refers to a single solvent, which may be organic, aqueous, and mixtures thereof. The term “organic solvent” means a solvent conventionally understood as such in the art, including polar organic solvents, non-polar organic solvents, water-miscible organic solvents, water immiscible organic solvents, and mixtures thereof.

Preferably, the solvent used in the dealkylation reaction is a solvent in which non-polar or hydrophobic compounds are preferentially or substantially soluble. Although not required, if desired, a nucleophilic agent can be added to promote or accelerate dealkylation.

Table 1 illustrates the effect of various solvents and conditions on the rate of dealklation of Rocuronium bromide I, to produce (2β, 3α, 5α, 16β, 17β)-17-acetoxy-3-hydroxy-2-(4-morpholinyl)-16(1-pyrrolidinyl)-androstane II. Two additional by-products (III and IV) have been identified in the reaction mixture and also are mentioned in Table 1.

The reactions illustrated in Table 1 are carried out using either the indicated solvent alone or in combination with sodium thiophenol, a nucleophilic reagent that reportedly promotes dealkylation by a transalkylation mechanism.

TABLE 1

| | | | % of II | % of I | % of III and |
|---|---|---|---|---|---|
| No. | Solvent | Reaction conditions | by HPLC | by HPLC | IV by HPLC |
| 1 | N,N-dimethylformamide | 24 g of (I) in 72 ml | 99.5% | 0.2% | III - NF |
| | (“DMF”) | DMF, 5 hours reflux | | | IV - 0.13% |
| 2 | Dimethylsulfoxide | 0.4 g of (I) in 12 ml | 95.0% | — | ND |
| | (“DMSO”) | DMSO, 6 hours reflux | | | |
| 3 | N,N-dimethylacetamide | 1 g of (I) in 5 ml DMA, | 87.4% | 7.4% | III - 0.3% |
| | (“DMA”) | 3 hours reflux | | | IV - 0.3% |
| 4 | Methylethyl ketone | 2 g of (I) in 60 ml | 39.2% | 54.1% | III - NF |
| | | methylethylketone 16 | | | IV - 0.4% |
| | | hours reflux | | | |
| 5 | Methylethyl ketone | 1 g of (I) in 66 ml | 14.4% | 80% | III - NF |
| | | methylethylketone + 0.87 g | | | IV - 4.4% |
| | | sodium | | | |
| | | thiophenol, 5 hours | | | |
| | | heating to 70° C. | | | |
| 6 | 2-propanol | 0.5 g of (I) in 16 ml 2- | 0.8% | 98.4% | ND |
| | | propanol 6 hours | | | |
| | | reflux | | | |
| 7 | 2-propanol | 1 g of (I) in 66 ml 2- | 14.1% | 21.7% | III - 0.5% |
| | | propanol + 0.87 g | | | IV - 67.3% |
| | | sodium thiophenol, 4 | | | |
| | | hours reflux | | | |
| 8 | Diethylamine | 0.5 g of (I) in 16 ml | 14.9% | 78.7% | ND |
| | | diethyl-amine, 6 | | | |
| | | hours reflux | | | |
| 9 | Methylisobutyl ketone | 0.4 g of (I) in 12 ml | 13.5% | 79.1% | ND |
| | | methylisobutyl-ketone | | | |
| | | 6 hours reflux | | | |

NF = not found,
ND = not determined

As can be seen from Table 1, the dealkylation of Rocuronium bromide in accordance with the present invention can be carried out under a variety of different solvent conditions, in the absence or presence of a nucleophilic agent. The use of sodium thiophenol is not preferred when the solvent is methylethyl ketone (entry 5) or 2-propanol (entry 7), as the sodium thiophenol does not appear to have a positive impact on the dealkylation when such solvents are used.

The dealkylation can be conducted at any suitable temperature. For example, Rocuronium bromide can be heated as a solution in DMF at a temperature of about 153° C., under reflux conditions, to achieve dealkylation at a conversion higher than 99% within 5 hours. Alternatively, Rocuronium bromide can be heated in DMF at a temperature of 70° C., to achieve dealkylation at a conversion of 44% within 5 hours.

Any suitable solvent quantity can be used in the dealkylation reaction. For Rocuronium bromide, the ratio of Rocuronium bromide to solvent in the alkylation step can be at least about 1 g/50 ml, at least about 1 g/20 ml, at least about 1 g/5 ml or at least about 1 g/3 ml.

Preferably, the product obtained by dealkylating Rocuronium bromide, (2β, 3α, 5α, 16β, 17β)-17-acetoxy-3-hydroxy-2-(4-morpholinyl)-16(1-pyrrolidinyl)-androstane II, has a purity of at least about 95%, and more preferably has a purity of at least about 97%, and still more preferably has a purity of at least about 99%, e.g., 99.5%.

In accordance with the present invention, the product obtained by dealkylating Rocuronium bromide, (2β, 3α, 5α, 16β, 17β)-17-acetoxy-3-hydroxy-2-(4-morpholinyl)-16(1-pyrrolidinyl)-androstane II, can be isolated from the reaction mixture by extraction using any suitable solvent. Suitable extraction solvents can include water-immiscible solvents such as, for example, ethyl acetate, diethyl ether, dipropyl ether, diisopropyl ether, dichloromethane, and the like, and mixtures thereof.

Any suitable drying agent can be used for drying the dealkylation product solution obtained by extraction from the reaction mixture. Suitable drying agents include one or more solid drying materials selected from a group of drying agents containing magnesium sulfate, sodium sulfate, calcium chloride, molecular sieves, and the like, and mixtures thereof. Magnesium sulfate is one of the preferred agents for drying an organic solvent solution of (2β, 3α, 5α, 16β, 17β)-17-acetoxy-3-hydroxy-2-(4-morpholinyl)-16(1-pyrrolidinyl)-androstane II.

In accordance with the present invention, the dealkylated product can be purified using any suitable process. For example, (2β, 3α, 5α, 16β, 17β)-17-acetoxy-3-hydroxy-2-(4-morpholinyl)-16(1-pyrrolidinyl)-androstane II can be purified by precipitation, crystallization or both. In one embodiment, (2β, 3α, 5α, 16β, 17β)-17-acetoxy-3-hydroxy-2-(4-morpholinyl)-16(1-pyrrolidinyl)-androstane II is crystallized by a process, which comprises the steps of:
a) dissolving the product in a suitable organic solvent, optionally at elevated temperature;
b) allowing the solution to cool sufficiently to produce crystals;
c) collecting the crystals by filtration and washing the crystals with cold solvent; and
d) drying the crystals, optionally at elevated temperature.

Non-limiting examples of solvents that may be used for crystallizing (2β, 3α, 5α, 16β, 17β)-17-acetoxy-3-hydroxy-2-(4-morpholinyl)-16(1-pyrrolidinyl)-androstane II include acetone, acetonitrile, and mixtures thereof.

Preferably, the obtained crystallized product (2β, 3α, 5α, 16β, 17β)-17-acetoxy-3-hydroxy-2-(4-morpholinyl)-16(1-pyrrolidinyl)-androstane II has a purity of at least about 95%, and more preferably has a purity of at least about 97%, and still more preferably has a purity of at least about 99%, and most preferably has a purity of at least about 99.5%, e.g., 99.9% or higher.

In accordance with the present invention, the purified (2β, 3α, 5α, 16β, 17β)-17-acetoxy-3-hydroxy-2-(4-morpholinyl)-16(1-pyrrolidinyl)-androstane II can be converted into Rocuronium bromide I using any suitable method. Preferably, the Rocuronim bromide thus produced is isolated from the reaction as a stable, powdered, non-hygroscopic solid, in substantially pure form. In a preferred embodiment, the Rocuronium bromide is produced by a process comprising the steps of:
a) reacting (2β, 3α, 5α, 16β, 17β)-17-acetoxy-3-hydroxy-2-(4-morpholinyl)-16(1-pyrrolidinyl)-androstane II with an excess of allyl bromide in the presence of a suitable solvent;
b) pouring the reaction mixture into an anti-solvent with stirring, to produce a wet precipitate comprising Rocuronium bromide;
c) isolating the wet precipitate in a pure form;
d) drying, spray-drying or lyophilizing the wet precipitate to produce a dried product;
e) dissolving the dried product in a buffered aqueous solution;
f) removing volatiles from the buffered aqueous solution; and
g) collecting the Rocuronium bromide as a substantially pure dry product.

The foregoing method results in a stable, dry, powdered and non-hygroscopic substantially pure Rocuronium bromide that is suitable as a raw material for producing pharmaceutical formulations of Rocuronium bromide for injection.

Accordingly, the method of the present invention provides an industrially viable process for recovering highly pure Rocuronium bromide from contaminated production batches. The method of the present invention also can be used for recovering other quaternary ammonium-containing neuromuscular blocking agents in highly pure form from contaminated production batches.

### EXAMPLES

The following examples further illustrate the invention but, of course, should not be construed as in any way limiting its scope. Products prepared according to the examples were analyzed as follows.

Analysis by High Performance Liquid Chromatography (HPLC):

High performance liquid chromatography (HPLC) was performed using the following conditions:
Column and packing—Inertsil ODS-2 Silica 250×4.6 mm, GL Science
UV Detection at 210 nm, 254 nm
Flow rate: 1.2 ml/min
Injection volume: 10 μl
Mobile phase: 25% Buffer, 0.01 M ammonium acetate, adjusted to pH 9 with ammonia, 75% methanol
Temperature: 22° C.

Analysis by Mass Spectrometry:

| Source Type: | ESI |
|---|---|
| Capillary temperature (° C.): | 200 |
| Sheath Gas Flow: | 60.0 |
| Aux Gas Flow: | 50.0 |
| Mode: | Positive polarity |
| Source voltage (kV): | 4.5 |
| Source current (uA): | 80.0 |
| capillary voltage (V): | 20.0 |

### Example 1

This example demonstrates a process for dealkylating Rocuronium bromide.

Rocuronium bromide (23.5 g) was placed in a 1000 ml three-necked flask, equipped with nitrogen inlet and reflux condenser. DMF was added (72 ml) and the mixture was refluxed for 5 hours during which time a black solution was obtained. The solution was allowed to cool to room temperature, dichloromethane was added (180 ml) and water (180 ml) and the mixture was stirred for about half an hour. Then, the layers were separated and a 1 ml sample was withdrawn from the organic layer, diluted with acetonitrile and analyzed by HPLC. According to HPLC chromatogram, the sample consisted of 99.5% (2β, 3α, 5α, 16β, 17β)-17-acetoxy-3-hydroxy-2-(4-morpholinyl)-16(1-pyrrolidinyl)-androstane, 0.2% Rocuronium bromide and 0.13% of (2β, 3α, 5α, 16β, 17β)-2-(4-morpholinyl)-16(1-pyrrolidinyl)-androstane-3,17-diol III.

The organic layer was dried over magnesium sulfate and evaporated to obtain 13.06 g of (2β, 3α, 5α, 16β, 17β)-17-acetoxy-3-hydroxy-2-(4-morpholinyl)-16(1-pyrrolidinyl)-androstane II in 67.5% yield. MS: m/z=489.4 [MH⁺]

### Example 2

This example demonstrates a process for dealkylating Rocuronium bromide.

Rocuronium bromide (3 g) was placed in a 100 ml three-necked flask equipped with a nitrogen inlet and a reflux condenser. DMF was added (15 ml) and the mixture was refluxed for 5 hours during which time a black solution was obtained. The solution was allowed to cool to room temperature and a 1 ml sample was withdrawn from the reaction mixture and evaporated at reduced pressure. Acetonitrile was added to the obtained solid to form a solution, out of which a sample of 20 μl was withdrawn and analyzed by HPLC.

According to the HPLC chromatogram, the sample consisted of 87.4% (2β, 3α, 5α, 16β, 17β)-17-acetoxy-3-hydroxy-2-(4-morpholinyl)-16(1-pyrrolidinyl)-androstane II, 7.3% Rocuronium bromide I, 0.3% of (2β, 3α, 5α, 16β, 17β)-2-(4-morpholinyl)-16(1-pyrrolidinyl)-androstane-3,17-diol III and 0.3% 1-[(2β, 3α, 5α, 16β, 17β)-2-(4-morpholinyl)-androstan-16-yl-]-1-(2-propenyl)-pyrrolidinium bromide-3,17-diol IV.

### Example 3

This example demonstrates a solventless process for dealkylating Rocuronium bromide.

Rocuronium bromide (4.5 g) was placed in an oven in a small flask and heated under vacuum at 150° C. for 10 hours during which time a dark red color was obtained. The material was allowed to cool to room temperature and a 25 mg sample was withdrawn, diluted with acetonitrile and analyzed by HPLC. According to the HPLC chromatogram, the sample consisted of 81% (2β, 3α, 5α, 16β, 17β)-17-acetoxy-3-hydroxy-2-(4-morpholinyl)-16(1-pyrrolidinyl)-androstane II and 16.4% Rocuronium bromide. The weight of the solid was 4 g.

### Example 4

This example demonstrates a process for dealkylating Rocuronium bromide.

Rocuronium bromide (3 g) was placed in a 100 ml three-necked flask, equipped with a nitrogen inlet and a reflux condenser, and DMA was added (15 ml). The mixture was refluxed for 3 hours during which time a black solution was obtained. The solution was allowed to cool to room temperature and a 1 ml sample was withdrawn from the reaction mixture and evaporated in vacuum. Acetonitrile was added to the obtained solid to form a solution, out of which a sample of 20 μl was withdrawn and analyzed by HPLC.

According to the HPLC chromatogram, the sample consisted of 91.5% (2β, 3α, 5α, 16β, 17β)-17-acetoxy-3-hydroxy-2-(4-morpholinyl)-16(1-pyrrolidinyl)-androstane II, 1.1% Rocuronium bromide I, 4.1% of (2β, 3α, 5α, 16β, 17β)-2-(4-morpholinyl)-16(1-pyrrolidinyl)-androstane-3,17-diol III and 1.4% 1-[(2β, 3α, 5α, 16β, 17β)-2-(4-morpholinyl)-androstan-16-yl-]-1-(2-propenyl)-pyrrolidinium-bromide-3,17-diol IV.

### Example 5

This example demonstrates the purification of a dealkylation product.

(2β, 3α, 5α, 16β, 17β)-17-acetoxy-3-hydroxy-2-(4-morpholinyl)-16(1-pyrrolidinyl)-androstane II (13.06 gram), obtained by dealkylating Rocuronium bromide, was suspended in acetonitrile (327 ml) and heated at 65° C. to obtain a clear solution. The solution was allowed to cool to room temperature and then to about 4° C. The crystals were filtered off, washed 2 times with cold acetonitrile (33 ml each) and dried at elevated temperature. 25 mg of the obtained crystals were dissolved in 25 ml of acetonitrile and injected to the HPLC. According to the HPLC chromatogram, the sample consisted of 99.9% of (2β, 3α, 5α, 16β, 17β)-17-acetoxy-3-hydroxy-2-(4-morpholinyl)-16(1-pyrrolidinyl)-androstane II, and no Rocuronium bromide was detected in the sample. Yield: 8.04 g, 61.5%.

### Example 6

This example demonstrates the conversion of a purified dealkylated product into a quaternary ammonium-containing neuromuscular blocking agent.

A mixture of (2β, 3α, 5α, 16β, 17β)-17-acetoxy-3-hydroxy-2-(4-morpholynyl)-16-(1-pyrrolidinyl)androstane II (7 g), allyl bromide (21 ml) and acetonitrile (28 ml) was stirred at room temperature for 3 hours. The solution was gradually poured into vigorously stirred isobutyl acetate (336 ml). The precipitated Rocuronium bromide was washed twice with isobutyl acetate (34 ml each), filtered off and dried overnight in a vacuum oven at 30° C. Yield: 8.18 gram, 93.7%.

According to the HPLC chromatogram, the sample consisted of 99.86% Rocuronium bromide and 0.03% of (2β, 3α, 5α, 16β, 17β)-17-acetoxy-3-hydroxy-2-(4-morpholynyl)-16-(1-pyrrolidinyl)androstane II.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for preparing a neuromuscular blocking agent, the method comprising dealkylating a neuromuscular blocking agent containing at least one quaternary ammonium group, to produce a dealkylated product, purifying the dealkylated product, to produce a purified dealkylated product, and converting the purified dealkylated product into a neuromuscular blocking agent, wherein the neuromuscular blocking agent is selected from the group consisting of Tubocurarine chloride, Pancuronium bromide, Vecuronium bromide, Rocuronium bromide and Atracurium besylate.

2. The process according to claim 1, comprising dealkylating Rocuronium bromide, to produce a dealkylated product, which is (2β, 3α, 5α, 16β, 17β)-17-acetoxy-3-hydroxy-2-(4-morpholinyl)-16(1-pyrrolidinyl)-androstane II, purifying the dealkylated product, and converting the purified dealkylated product into Rocuronium bromide.

3. The process according to claim 2, wherein the neuromuscular blocking agent is dealkylated by heating.

4. The process according to claim 3, wherein the neuromuscular blocking agent is dealkylated in the presence of a solvent.

5. The process according to claim 2, comprising the steps of:
a) heating Rocuronium bromide, to obtain (2β, 3α, 5α, 16β, 17β)-17-acetoxy-3-hydroxy-2-(4-morpholinyl)-16(1-pyrrolidinyl)-androstane II;
b) precipitating the (2β, 3α, 5α, 16β, 17β)-17-acetoxy-3-hydroxy-2-(4-morpholinyl)-16(1-pyrrolidinyl)-androstane II from an organic solvent, to obtain a purified product;
c) optionally crystallizing the (2β, 3α, 5α, 16β, 17β)-17-acetoxy-3-hydroxy-2-(4-morpholinyl)-16(1-pyrrolidinyl)-androstane II, to obtain a purified product;
d) converting the purified (2β, 3α, 5α, 16β, 17β)-17-acetoxy-3-hydroxy-2-(4-morpholinyl)-16(1-pyrrolidinyl)-androstane II into Rocuronium bromide;
e) isoloating the Rocuronium bromide in substantially pure form.

6. The process according to claim 2, comprising the steps of:
a) heating Rocuronium bromide, optionally in an organic solvent, to dealkylate the Rocuronium bromide;
b) adding water and a substantially water-immiscible organic solvent, mixing to extract the dealkylated product into the organic layer and separating the layers; and
c) drying and evaporating the organic layer, to produce a purified form of (2β, 3α, 5α, 16β, 17β)-17-acetoxy-3-hydroxy-2-(4-morpholinyl)-16(1-pyrrolidinyl)-androstane II.

7. The process according to claim 6, wherein the Rocuronium bromide is dealkylated in a solvent selected from the group consisting of N,N-dimethylformamide (DMF), N,N-dimethylacetamide (DMA) and N,N-dimethylsulfoxide (DMSO), or a mixture thereof.

8. The process according to claim 6, wherein the Rocuronium bromide is dealkylated at temperature in the range of from about 25° C. to about 180° C.

9. The process according to claim 6, wherein the Rocuronium bromide is dealkylated at temperature in the range of from about 150° C. to about 180° C.

10. The process according to claim 6, wherein the Rocuronium bromide is dealkylated by refluxing in a solvent.

11. The process according to claim 6, wherein the ratio of Rocuronium bromide to solvent in the dealkylation step is at least about 1 g/50 ml.

12. The process according to claim 6, wherein the ratio of Rocuronium bromide to solvent in the dealkylation step is at least about 1 g/20 ml.

13. The process according to claim 6, wherein the ratio of Rocuronium bromide to solvent in the dealkylation step is at least about 1 g/5 ml.

14. The process according to claim 6, wherein the ratio of Rocuronium bromide to solvent in the dealkylation step is at least about 1 g/3 ml.

15. The process according to claim 6, wherein the solvent used for extracting the dealkylated product from the reaction solution is selected from the group consisting of ethyl acetate, diethyl ether, dipropyl ether, diisopropyl ether, and dichloromethane, or a mixture thereof.

16. The process according to claim 6, wherein the organic layer is dried by mixing with a drying effective amount of a solid drying material selected from the group consisting of magnesium sulfate, sodium sulfate, calcium chloride, and molecular sieves, or a mixture thereof.

17. The process according to claim 6, wherein the purified (2β, 3α, 5α, 16β, 17β)-17-acetoxy-3-hydroxy-2-(4-morpholinyl)-16(1-pyrrolidinyl)-androstane II has a purity of at least about 99%.

18. The process according to claim 6, wherein the purified (2β, 3α, 5α, 16β, 17β)-17-acetoxy-3-hydroxy-2-(4-morpholinyl)-16(1-pyrrolidinyl)-androstane II has a purity of at least 99.5%.

19. The process according to claim 2, wherein the (2β, 3α, 5α, 16β, 17β)-17-acetoxy-3-hydroxy-2-(4-morpholinyl)-16(1-pyrrolidinyl)-androstane II is purified by crystallization, wherein the crystallization comprises:
a) dissolving the product in an organic solvent, optionally at elevated temperature;
b) allowing the solution to cool, to produce crystals;
c) collecting the crystals by filtration and washing the crystals; and,
d) drying the crystals, optionally at elevated temperature.

20. The process according to claim 19, wherein the crystallization is performed in a solvent selected from the group consisting of acetone and acetonitrile, or a mixture thereof.

21. The process according to claim 20, wherein the crystallized product (2β, 3α, 5α, 16β, 17β)-17-acetoxy-3-hydroxy-2-(4-morpholinyl)-16(1-pyrrolidinyl)-androstane II has a purity of at least about 99%.

22. The process according to claim 20, wherein the crystallized product (2β, 3α, 5α, 16β, 17β)-17-acetoxy-3-hydroxy-2-(4-morpholinyl)-16(1-pyrrolidinyl)-androstane II has a purity of at least 99.9%.
